(19) United States
(12) Patent Application Publication
(10) Pub. No.: US 2006/0196966 A1
(43) Pub. Date: Sep. 7, 2006

(54) Taste enhancer

(76) Inventor: Dido Cheng, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/401,256

(22) Filed: Apr. 11, 2006

Related U.S. Application Data
(63) Continuation-in-part of application No. 10/834,875, filed on Apr. 30, 2004.

(30) Foreign Application Priority Data
Apr. 11, 2005 (TW) 094111393

(51) Int. Cl. A61L 9/04

(52) U.S. Cl. 239/60

(57) ABSTRACT

This invention relates to a taste enhancer, which comprises a connecting member, an extension member and an aroma-producing device. The connecting member is connected to a rim of a food/drink container and the extension member is connected to the connecting member and extends to a particular location, which is near a user's nose when the user consumes food/drink from the food/drink container. Furthermore, the aroma-producing device is fixed to the extension member so as to produce a specific aroma. Therefore, the user smells the specific aroma when consumes food/drink from the food/drink container that changes the user's perception of the food/drink by the specific aroma.

### TECHNICAL FIELD

This invention relates generally to a taste enhancer, which is provided for enhancing a single flavor food or drink to have particular flavors.

### BACKGROUND OF THE INVENTION

Nowadays, people change their diet because they realize that fine food and fast food with high calorie and cholesterol can threaten their health; therefore, organic and health foods have become popular. Organic and health foods, such as diet foods and drinks or natural food, are generally unsavory. Furthermore, it goes without saying that consuming proper amount of water is good for health but some people drink a great amount of flavored drinks instead of water because water is tasteless. Similarly, flavored drinks consisting of sugar and artificial ingredients are useless to their bodies.

In addition, food or drink containers with special or attractive designs and shapes can only enhance visual attraction for users and the users would have special feelings to the food or drink while using the containers. However, these designed and shaped containers mainly provide visual effects but not flavors to the users.

Therefore, on the basis of foregoing health considerations, to provide people with enhanced taste to particular drinks and foods is in demand.

### SUMMARY OF INVENTION

The primary objective of the present invention is to provide a special taste object that releases a specific aroma used to stimulate appetite through the olfaction during meals.

More specifically, according to research, human being's taste sense is not completely derived from the tongue only, but also from palate and olfaction combined. Basically, the tongue can only distinguish four tastes: sourness, sweetness, bitterness, and saltiness. Out of the many savors that we come upon regularly, the four tastes of sourness, sweetness, bitterness, and saltiness are transmitted to the brain through the tongue, but as for the savories of chicken soup, barbeque, rice, fruits and etc. that comes from foods are transmitted to the brain through the nasal from the mouth, then the brain will determine the food according to its taste, so that a person determines the kind of food within the mouth even if the person is eating with eyes closed. For instance, when eating an apple, the sour-sweet taste is transmitted to the brain through the tongue, and then the brain will integrate the information and determine that the food within the mouth is an apple. Therefore, the present invention is competed through the application of this olfaction stimulant principle.

Substantially, the taste enhancer of the present invention mainly comprises a connecting member, an extension member and an aroma-producing device. The connecting member is connected to a food/drink container's rim and the extension member is connected to the connecting member and extends to a particular location, which is near a user's nose when the user consumes food/drink from the food/drink container. The aroma-producing device is fixed to the extension member for producing a specific aroma.

Therefore, when the user holds up the food/drink container to his/her mouth and consumes food/drink from it, the user smells the specific aroma released from the aroma-producing device, and thus the user's perception of the food/drink is changed by the specific aroma.

### BRIEF DESCRIPTION OF DRAWINGS

The invention will be more clearly understood after referring to the following detailed description read in conjunction with the drawings wherein:

FIG. 1 is an exploded view of a first embodiment of the present invention;

FIG. 2 is a cross sectional view of the first embodiment of the present invention;

FIG. 3 is a schematic view of the first embodiment of the present invention in use;

FIG. 4 is an exploded view of a second embodiment of the present invention;

FIG. 5 is an exploded view of a third embodiment of the present invention;

FIG. 6 is an exploded view of a fourth embodiment of the present invention;

FIG. 7 is a perspective view of a fifth embodiment of the present invention;

FIG. 8 is a perspective view of the fifth embodiment of the present invention;

FIG. 9 is an exploded view of a sixth embodiment of the present invention;

FIG. 10 is an exploded view of a seventh embodiment of the present invention;

FIG. 11 is an exploded view of an eighth embodiment of the present invention;

FIG. 12 is an exploded view of a ninth embodiment of the present invention;

FIG. 13 is a cross sectional view of the ninth embodiment of the present invention;

FIG. 14 is a partial sectional view of a tenth embodiment of the present invention;

FIG. 15 is a perspective view of an eleventh embodiment of the present invention;

FIG. 16 is an exploded view of a twelfth embodiment of the present invention;

FIG. 17 is an exploded view of a thirteenth embodiment of the present invention; and

FIG. 18 is a perspective view of a fourteenth embodiment of the present invention.

FIG. 19 is a perspective view of a fifteenth embodiment of the present invention.

FIG. 20 is a perspective view of a sixteenth embodiment of the present invention.

FIG. 21 is a top view of the sixteenth embodiment of the present invention.

### DETAILED DESCRIPTION OF PREFERRED EMBODIMENTS

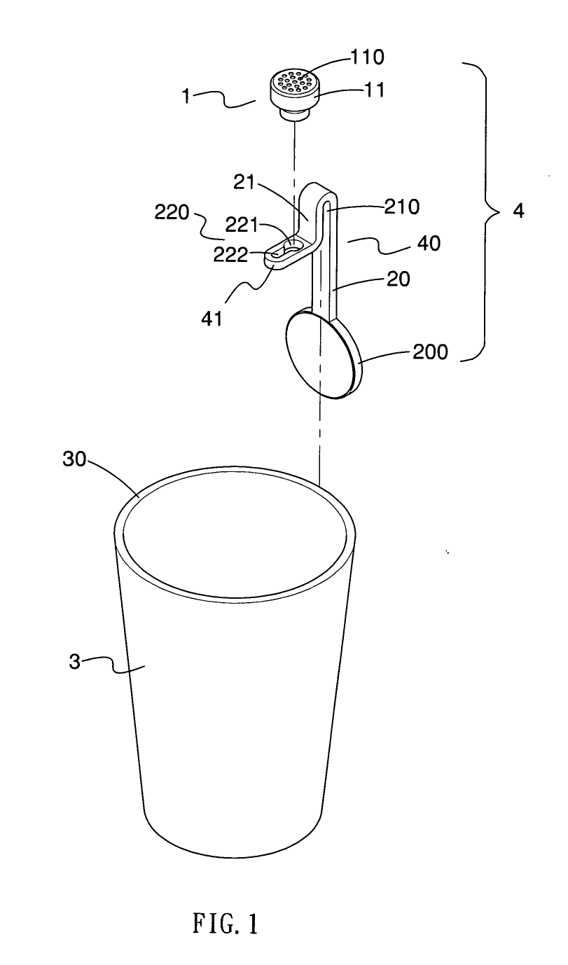

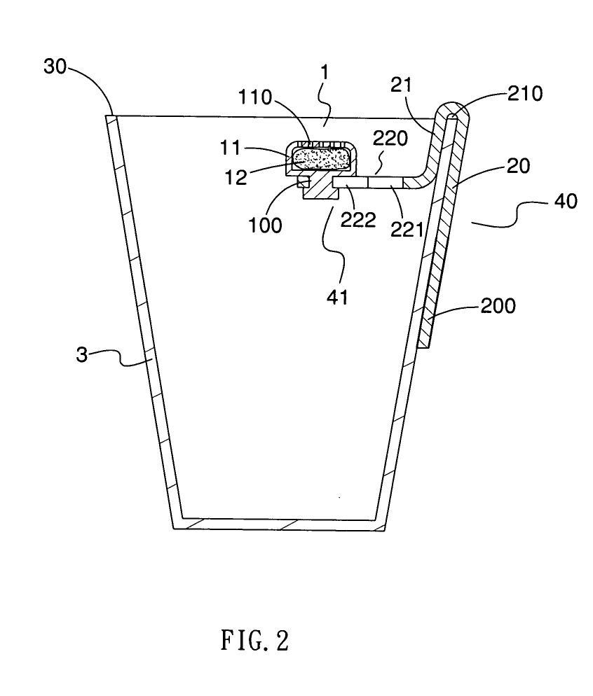

In following embodiments, the present invention is applied to cups but to those skilled in the art would know that the present invention can also be used in other similar food/drink containers, such as bowls, plates, and the like. FIGS. 1-2 show a taste enhancer 4 as a first embodiment of the present invention. The taste enhancer 4 is mainly used to be connected to a food/drink container, such as a cup 3, which has a rim 30 so as to form a food passage. In addition, the taste enhancer 4 comprises a connecting member 40, an extension member 41 and an aroma-producing device 1.

The connecting member 40 is provided for connecting to the cup 3. The extension member 41 has one end is connected to the connecting member 40 and another end extends to a particular location, which is near a user's nose when the user consumes food/drink from the cup 3. The aroma-producing device 1 is fixed to the extension member 41 so as to produce a specific aroma. Furthermore, the aroma-producing device 1 comprises a case 11 and an aroma-producer 12. The case 11 is attached to the extension member 41 and has a plurality of apertures 110 and a neck 100. The aroma producer 12 is installed within the case 11 for produce the specific aroma.

Furthermore, the connecting member 40 and the extension member 41 are formed by bending a flexible plate or molded integrally by a plastic material. The connecting member 40 further comprises an outer clip 20 and an inner clip 21 that forms a clip slot 210 for clipping onto the rim 30 of the cup 3. When the connecting member 40 is connected to the rim 30 of the cup 3 through the clip slot 210, the outer clip 20 is attached to an outer surface of the cup 3 and the inner clip 21 is attached to an inner surface of the cup 3. In addition, a bottom of the outer clip 20 has an attachment 200 with a larger plane provided for attaching to the outer surface of the cup 3 stably. More specifically, the attachment 200 can be arranged with an adhesive layer, a magnetic object, a reusable fastening tape or a suction disc on an attaching side of the attachment 200 so as to attach itself to the outer surface of the cup 3, whereas the other corresponding side can be used to print trademarks, logos or patterns thereon.

Referring to FIG. 2, the extension member 41 extends from one end of the inner clip 21 to the inside of the cup 3. In addition, the extension member 41 has an opening 220 to secure the aroma-producing device 1. The opening 220 is formed by a first slot 221 and a second slot 222, and the first and second slots 221, 222 are interconnected. Also, a diameter of the first slot 221 is larger than a diameter of the second slot 222 so that the neck 100 of the case 11 slips into the second slot 222 from the first slot 221 and is fixed to the second slot 222.

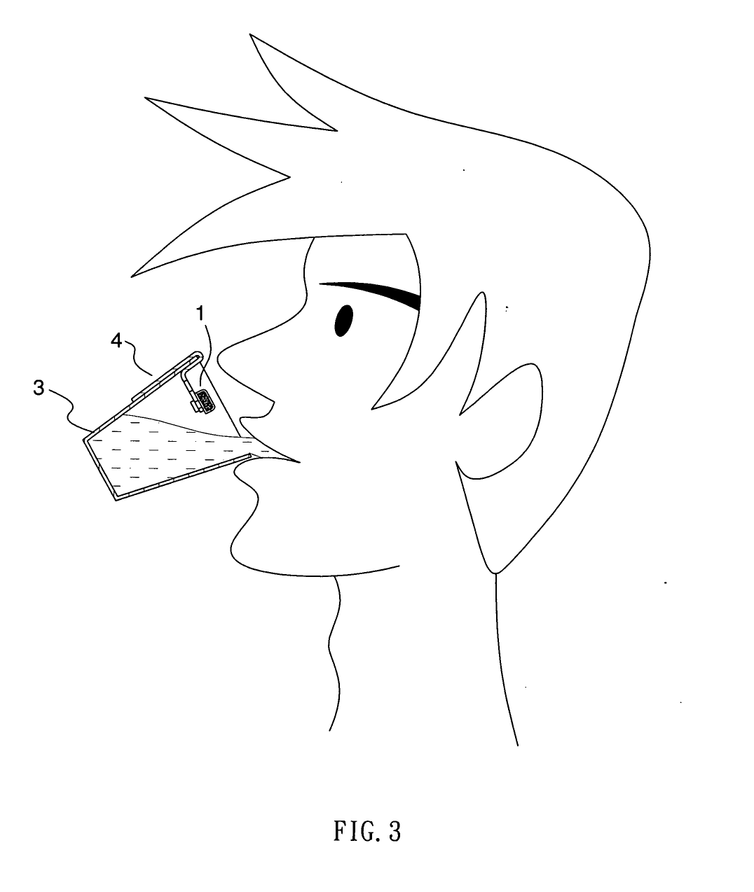

FIG. 3 shows the user is consuming the food/drink by using the cup 3 with the taste enhancer 4. When the user holds up the cup 3 to the mouth and drinks the fluid contained within the cup 3, the specific aroma released from the taste enhancer 4 will be carried directly to the user's nasal as the taste enhancer 4 moves near the user's nose. Therefore, as the user drinks the fluid from the cup 3, the user will continuously smell the specific aroma released from the taste enhancer 4 that creates a sense of drinking the fluid of the specific aroma.

For an example, if the cup 3 contains unflavored water and the taste enhancer 4 releases the aroma of lemon and is not in contact with the water, as the user drinks from the water, the aroma of lemon will continuously be inhaled into the user's nasal and transmitted to the user's brain. At the moment, the user drinks only water but by the stimulation of the olfactory, the user could sense the taste of lemon water and could even taste the sourness in the user's mouth.

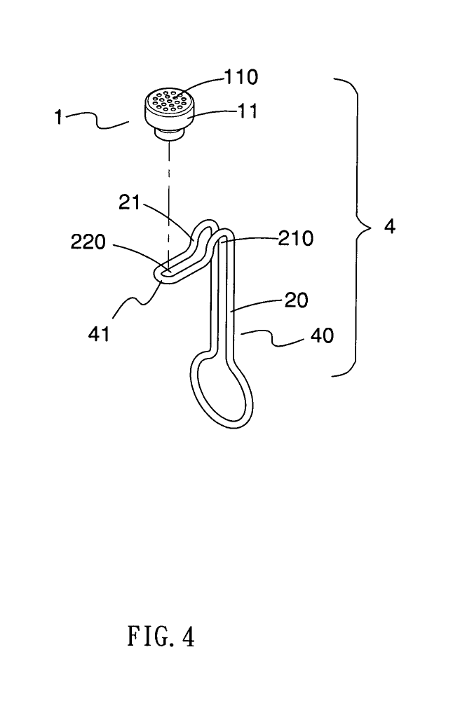

FIG. 4 shows a second embodiment of the present invention. The second embodiment is similar to that of the first embodiment and the main difference is that the connecting member 40 and the extension member 41 of the second embodiment is formed integrally by a bent steel wire or other bent metal wires.

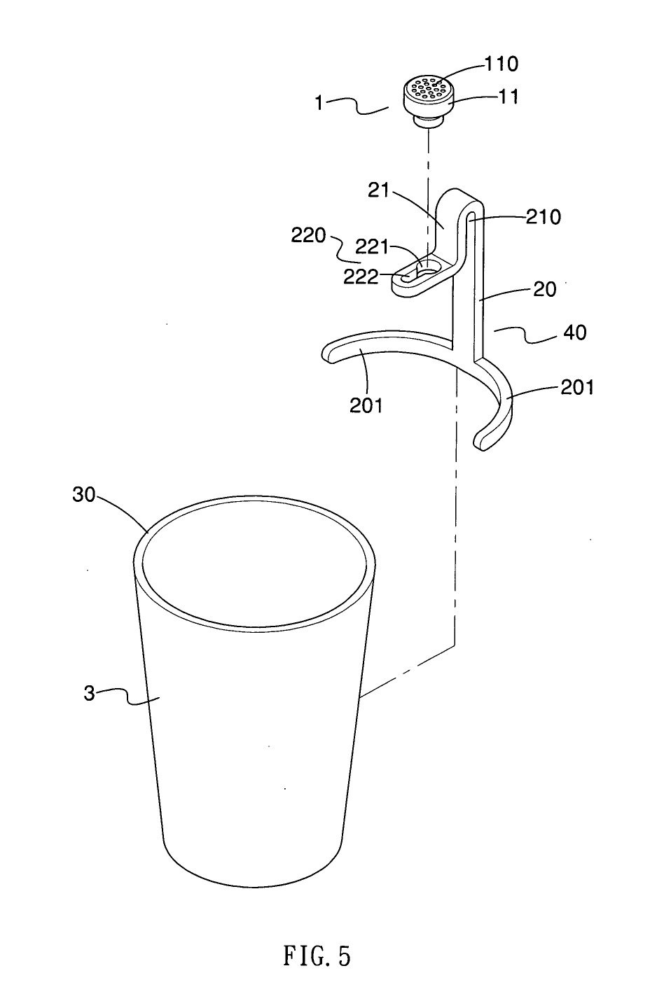

A third embodiment of the present invention is shown in FIG. 5. Similarly, the structure of the third embodiment is similar to that of the first embodiment and the main difference is that the outer clip 20 of the connecting member 40 comprises a gripping claw 201 at a bottom of the outer clip 20. The gripping claw 201 is used to clip onto the outer surface of the cup 3.

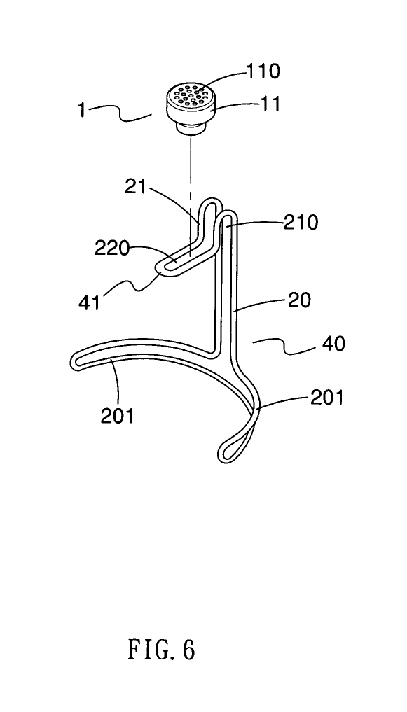

FIG. 6 shows a fourth embodiment of the present invention. The fourth embodiment is similar to the third embodiment and the main difference is that the connecting member 40 and the extension member 41 of the fourth embodiment is formed integrally by a bent steel wire or other bent metal wires.

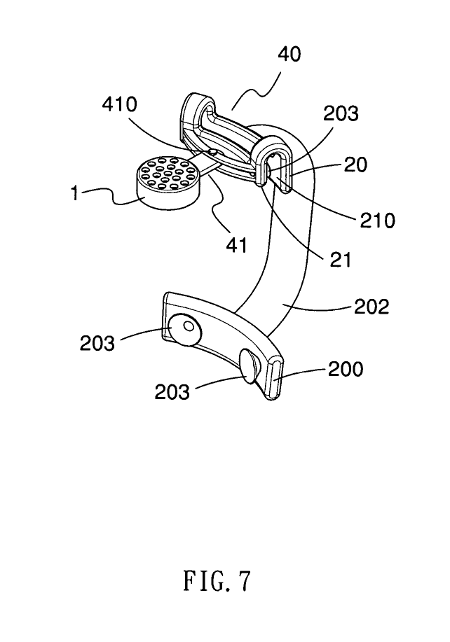

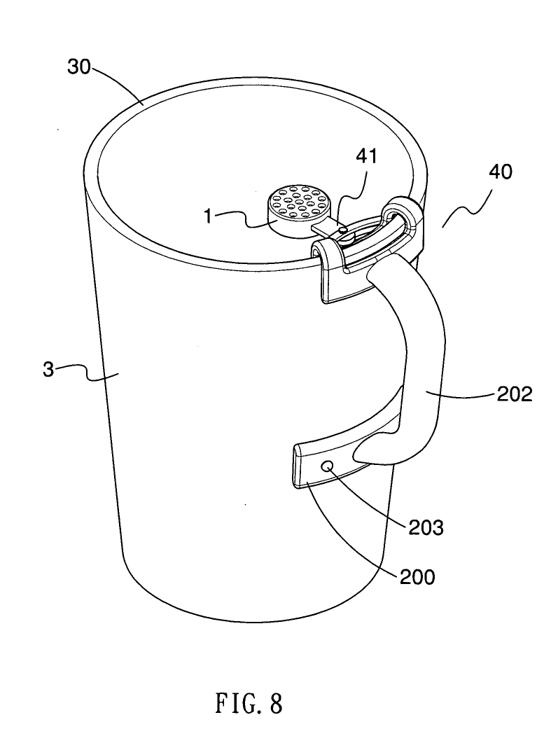

FIGS. 7-8 show a fifth embodiment of the present invention, which is similar to the first embodiment except the connecting member 40 and the extension member 41. The connecting member 40 is connected to the cup 3 and comprises the outer clip 20 and the inner clip 21 that form the clip slot 210, which is clipped onto the rim 30 of the cup 3. Furthermore, the outer clip 20 forms with a handle 202 for the user to grip and the bottom of the handle 202 has the attachment 200 for attaching to the outer surface of the cup 3. Similarly to the first embodiment, the attachment 200 also can be arranged with an adhesive layer, a magnetic object, a reusable fastening tape or a suction disc on an attaching side of the attachment 200 so as to attach itself to the outer surface of the cup 3. As shown in FIGS. 7-8, a plurality of suction cups 203 are arranged to the attachment 200 and the inner clip 21 as well for assisting in connection between the connecting member 40 and the cup 3. The extension member 41 is pivotally connected to the connecting member 40 and the aroma-producing device 1 is fixed to the extension member 41. Therefore, the aroma-producing device 1 is rotatable via the extension member 41. Preferably, the extension member 41 is pivoted to the connecting member 40 by a shaft 410.

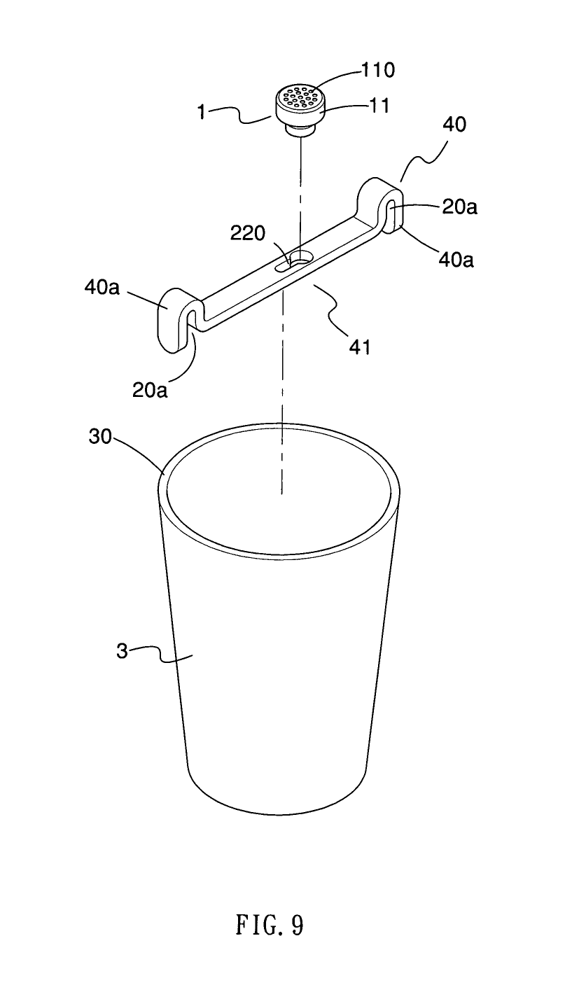

A sixth embodiment of the present invention is shown in FIG. 9. The sixth embodiment is similar to the first embodiment and the main difference is that the connecting member 40 comprises two clamps 40a. Each of the two clamps 40a has a clip slot 20a for clipping onto the rim 30 of the cup 3. Moreover, two ends of the extension member 41 are connected to the two clamps 40a respectively and an opening 220 is formed in the center of the extension member 41.

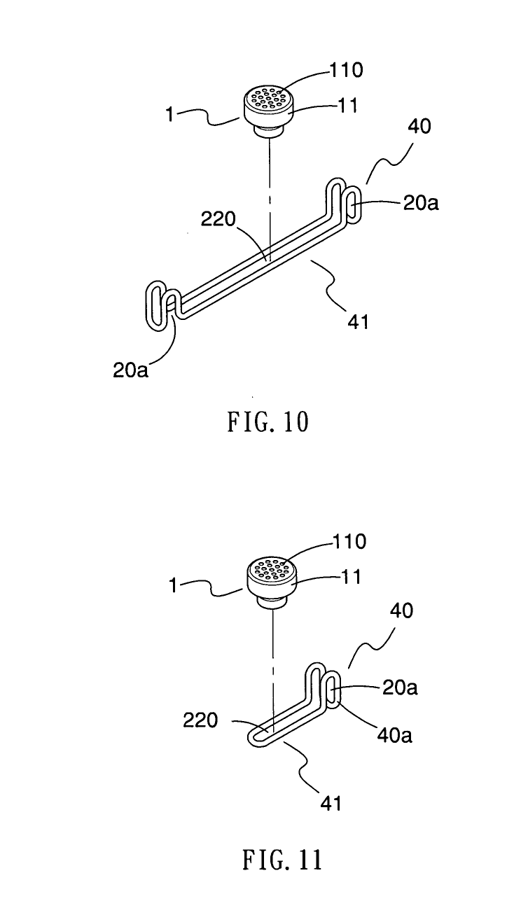

FIG. 10 shows a seventh embodiment of the present invention. The seventh embodiment is similar to the sixth embodiment and the main difference is that the connecting member 40 and the extension member 41 is formed by a bent wire.

FIG. 11 shows an eighth embodiment of the present invention. The eighth embodiment is similar to the sixth embodiment and the main difference is that the connecting member 40 of the seventh embodiment only has one clamp 40a.

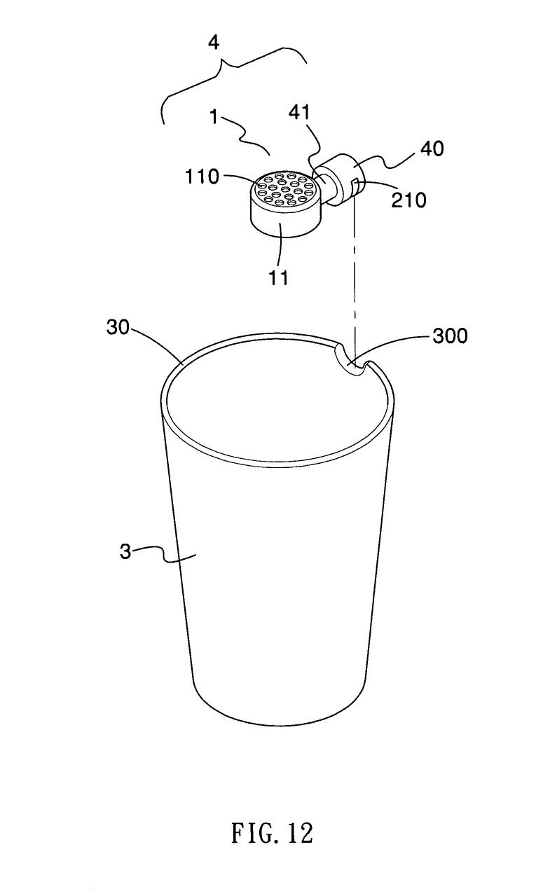

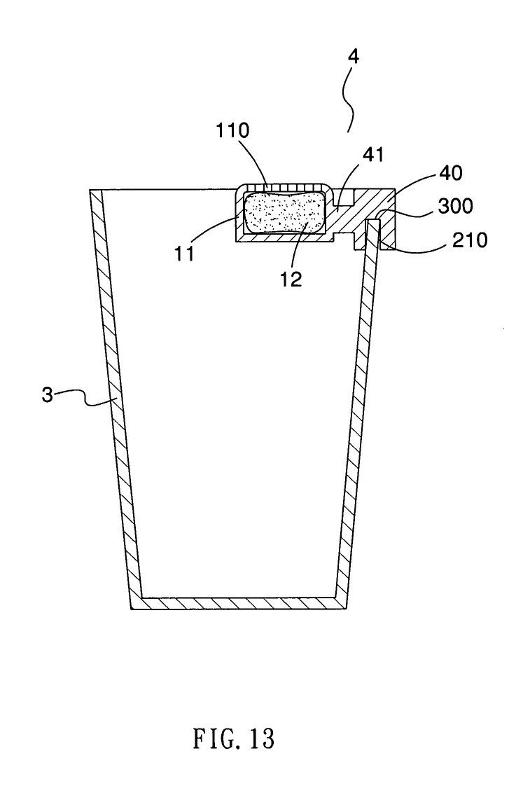

A ninth embodiment of the present invention is shown in FIGS. 12-13. The ninth embodiment mainly integrates the connecting member 40, the extension member 41 and the case 11 so as to form an integral taste enhancer 4. The integral taste enhancer 4 is provided for inserting to a recess 300 formed on the rim 30 of the cup 3 by the clip slot 210 of the connecting member 40 to directly integrate the taste enhancer 4 and the cup 3.

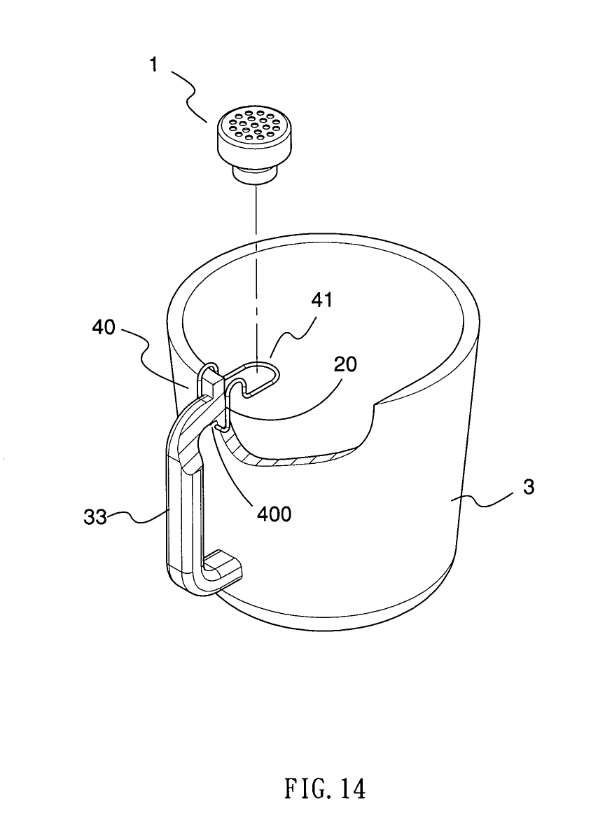

FIG. 14 shows a tenth embodiment of the present invention. The tenth embodiment is similar to the eighth embodiment and the main difference is that a bottom of the outer clip 20 of the connecting member 40 forms a gap 400, which enables the outer clip 20 to clip onto a handle 33 of the cup 3.

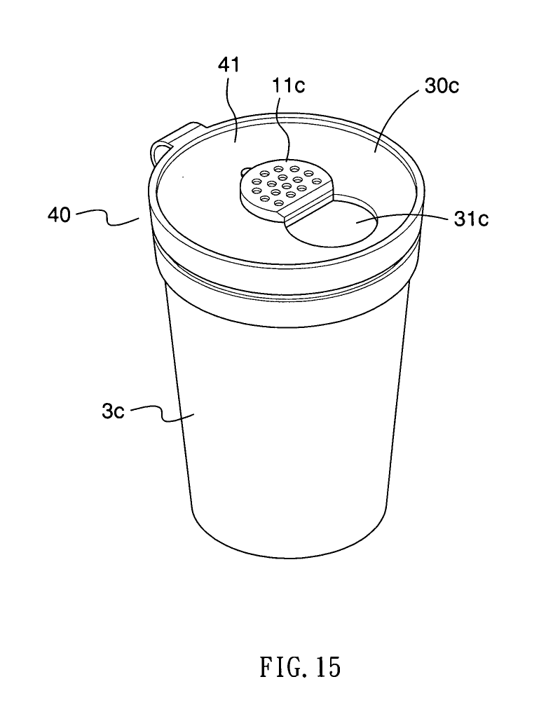

FIG. 15 shows an eleventh embodiment of the present invention. In the eleventh embodiment, the connecting member 40 is connected to the cup 3c and the extension member 41 comprises a cap 30c for covering the food/drink passage of the cup 3c. The cap 30c has a through hole 31c provided for receiving the case 11c.

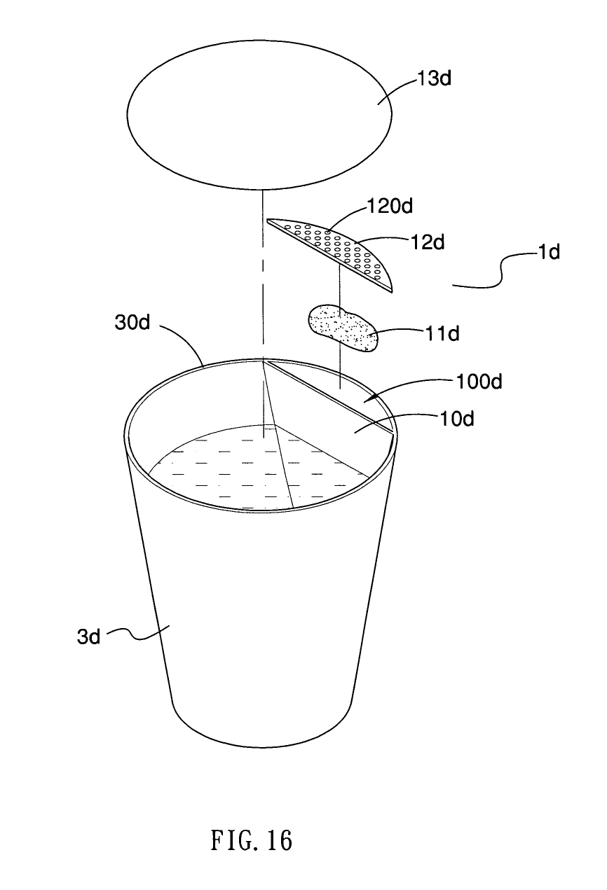

FIG. 16 shows twelfth embodiment of the present invention. The twelfth embodiment shows a food/drink container, which comprises a container body 3d and a taste enhancer 1d. The container body 3d has a rim 30d that forms a food passage. The taste enhancer 1d comprises a partition 10d and an aroma-producer 11d. The partition 10d is attached to the rim 30d of the container body 3d that forms a space 100d, which is near the user's nose when the user consumes food/drink from the container body 3d. In addition, the aroma-producer 11d is placed within the space 100d for providing the specific aroma. As shown in FIG. 16, the food/drink container of the twelfth embodiment further comprises a cover 12d and a sealing sheet 13d. The cover 12d is provided for covering the space 100d and has a plurality of apertures 120d for releasing the specific aroma. Moreover, the sealing sheet 13d is provided for covering the rim 30d of the container body 3d and the cover 12d. In the twelfth embodiment, the sealing sheet 13d can be sealed to the rim 30d of the container body 3d by adhesives or heat bonding processes.

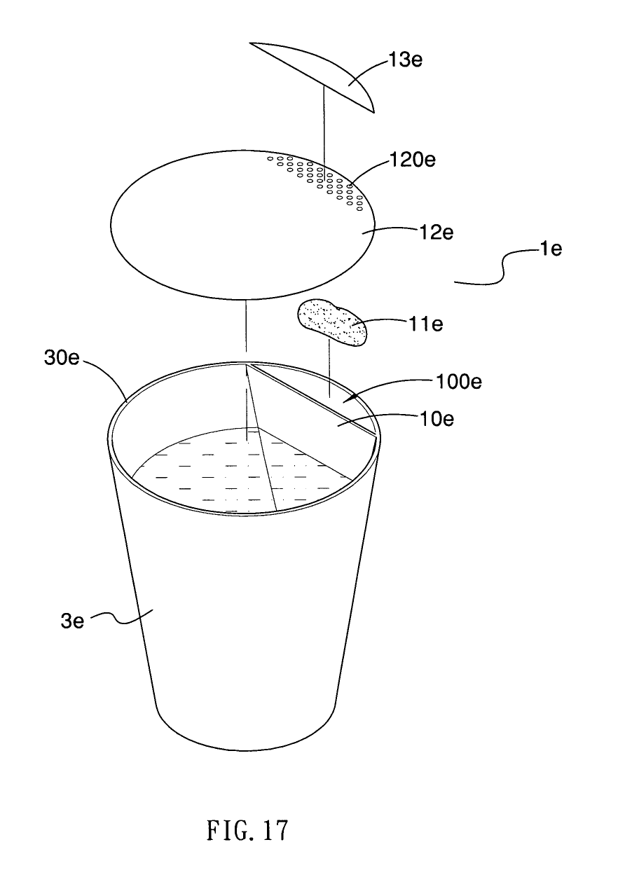

FIG. 17 shows a thirteenth embodiment of the present invention. The thirteenth embodiment is similar to the twelfth embodiment and comprises a container body 3e and a taste enhancer 1e. The container body 3e has a rim 30e to form a food passage. The taste enhancer 1e comprises a partition 10e and an aroma-producer 11e. The thirteenth embodiment of the present invention further comprises a sealing sheet 12e and a sealing layer 13e. The sealing sheet 12e is provided for covering the rim 30e of the container body 3e and the space 100e and the sealing sheet 12e corresponding to the space 100e has a plurality of apertures 120e. Moreover, the sealing layer 13e is provided for covering all the apertures 120e of the sealing sheet 12e.

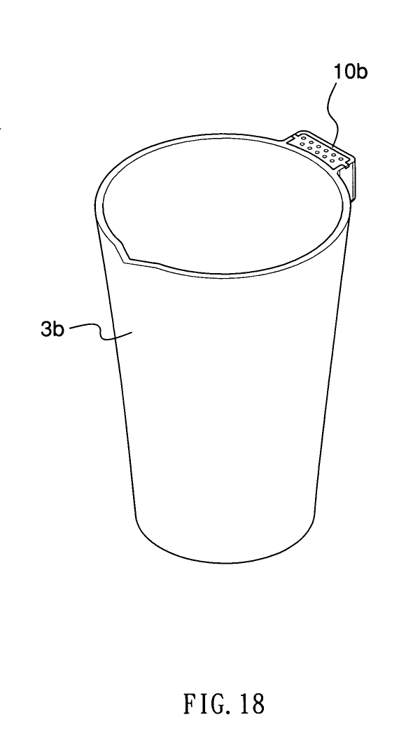

A fourteenth embodiment of the present invention is shown in FIG. 18. The fourteenth embodiment is similar to the twelfth embodiment except the position of the partition 10b.

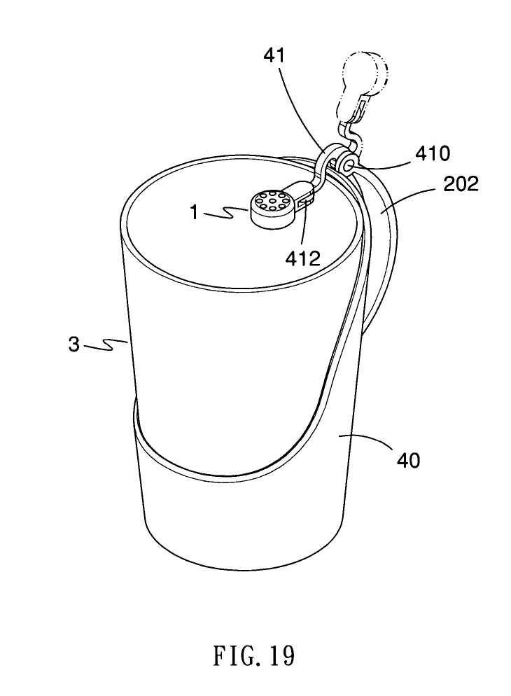

FIG. 19 shows a fifteenth embodiment of the present invention, which is similar to the first embodiment except the connecting member 40 and the extension member 41. In this embodiment, a cup holder 40 is used for serving as the connecting member 40 for carrying the cup 3. Furthermore, the connecting member 40, namely the cup holder, is formed with a handle 202 for the user to grip. The extension member 41 is pivotally connected to the connecting member 40 and the aroma-producing device 1 is pivotally connected to the extension member 41. Preferably, the extension member 41 is pivoted to the connecting member 40 by a shaft 410 and the aroma-producing device 1 is pivotally to the extension member 41 by a pivot 412, as shown in FIG. 19, so that the aroma-producing device 1 can be adjusted to a desire position close to the nose for comfortably tasting.

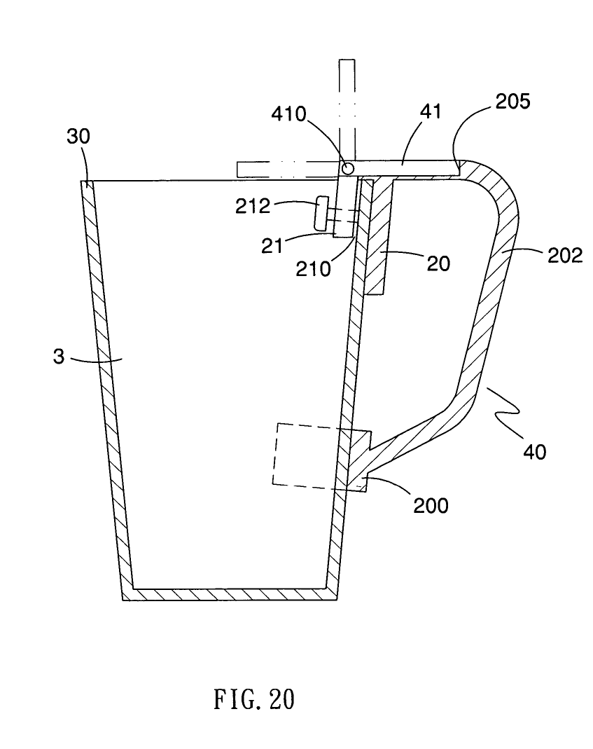

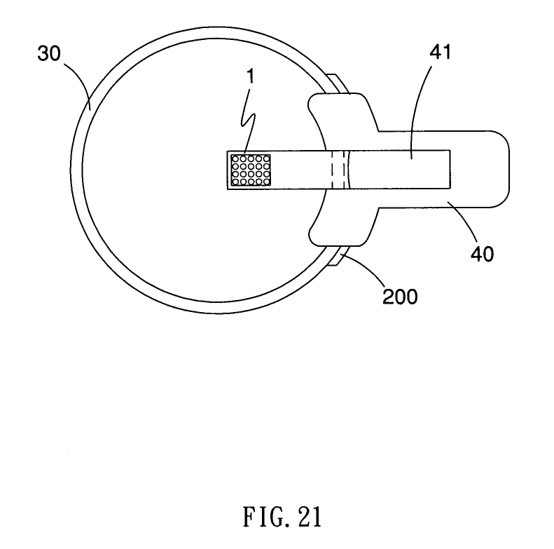

FIGS. 20-21 show a sixteenth embodiment of the present invention, which is similar to the fifth embodiment except the connecting member 40 and the extension member 41. The connecting member 40 is connected to the cup 3 and comprises the outer clip 20 and the inner clip 21 that form the clip slot 210, which is clipped onto the rim 30 of the cup 3. The clip slot 210 is arranged to a suitable dimension for available on cups with different sizes. Furthermore, the outer clip 20 forms with a handle 202 for the user to grip and the bottom of the handle 202 has the attachment 200 having a cambered surface against the outer surface of the cup 3 so as to support the cup 3. The cambered surface of the attachment 200 has a radian being arranged to a degree for embracing the outer surface of the cup 3, also suitable for cups with different sizes. The inner clip 21 can be secured to the inner surface of the cup 3 via a screw 212 or the like. The extension member 41 is pivotally connected to the connecting member 40. More particularly, an upper portion of the handle 202 is formed with a recess 205 for receiving the extension member 41. The aroma-producing device 1 is installed in an end of the extension member 41, as shown in FIG. 21. Therefore, the aroma-producing device 1 is movable via the extension member 41. Preferably, the extension member 41 is pivoted to the connecting member 40 by a shaft 410.

Numerous characteristics and advantages of the invention have been set forth in the foregoing description, together with details of the structure and function of the invention, and the novel features thereof are pointed out in appended claims. The disclosure, however, is illustrative only, and changes may be made in detail, especially, in matters of shape, size and arrangement of parts, materials and the combination thereof within the principle of the invention, to the full extent indicated by the broad general meaning of the terms in which the appended claims are expressed.

We claim:

1. A taste enhancer comprising:
a connecting member, for connecting to a food/drink container, the food/drink container having a rim so as to form a food passage;
an extension member, connected to the connecting member and extending to a particular location, the particular location is near a user's nose when the user consumes food/drink from the food/drink container; and
an aroma-producing device, fixed to the extension member so as to produce a specific aroma in the particular location.

2. The taste enhancer of claim 1, wherein the aroma-producing device comprises:
a case, attached to the extension member, the case having a plurality of apertures; and
an aroma-producer, installed within the case for producing the specific aroma.

3. The taste enhancer of claim 1, wherein the connecting member comprises an outer clip and an inner clip that form a clip slot for clipping onto the rim of the food/drink container, wherein the outer clip is provided for attaching to an outer surface of the food/drink container and the inner clip is provided for attaching to an inner surface of the food/drink container.

4. The taste enhancer of claim 3, wherein the extension member is extending from the inner clip and has an opening thereon to secure the aroma-producing device.

5. The taste enhancer of claims 3, wherein the outer clip of the connecting member has an attachment attached to the outer surface of the food/drink container.

6. The taste enhancer of claims 4, wherein the outer clip of the connecting member has an attachment attached to the outer surface of the food/drink container.

7. The taste enhancer of claims 3, wherein the outer clip of the connecting member comprises a gripping claw to clip onto the outer surface of the food/drink container.

8. The taste enhancer of claims 4, wherein the outer clip of the connecting member comprises a gripping claw to clip onto the outer surface of the food/drink container.

9. The taste enhancer of claim 1, wherein the connecting member comprises two clamps, each of the two clamps having a clip slot to clip onto the rim of the food/drink container; and
two ends of the extension member are connected to the two clamps respectively and a center of the extension member is formed with an opening.

10. The taste enhancer of claim 4, wherein the opening of the extension member is formed by a first slot and a second slot interconnected with the first slot, a diameter of the first slot being larger than a diameter of the second slot.

11. The taste enhancer of claim 9, wherein the opening of the extension member is formed by a first slot and a second slot interconnected with the first slot, a diameter of the first slot being larger than a diameter of the second slot.

12. The taste enhancer of claim 10, wherein the aroma-producing device comprises:
a case, having a neck for fixing to the second slot and a plurality of apertures; and
an aroma-producer, installed within the case for producing the specific aroma.

13. The taste enhancer of claim 2, wherein the extension member comprises a cap for covering the food passage of the food/drink container, the cap having a through hole thereon for receiving the case.

14. The taste enhancer of claim 3, wherein the extension member is pivotally connected to the connecting member, whereby the aroma-producing device is rotatable via the extension member.

15. The taste enhancer of claims 14, wherein the outer clip of the connecting member further forms with a handle for the user to grip and the handle has an attachment attached to the outer surface of the food/drink container.

16. A food/drink container comprising:
a container body, having a rim so as to form a food passage; and
a taste enhancer, comprising:
a partition, attached to the rim of the container body so as to form a space, wherein the space is near a user's nose when the user consumes drink/food from the container body; and
an aroma-producer, placed within the space for producing a specific aroma.

17. The food/drink container of claim 16, further comprising a cover for covering the space, the cover having a plurality of apertures.

18. The food/drink container of claim 17, further comprising a sealing sheet for covering the rim of the container body and the cover.

19. The food/drink container of claim 16, further comprising a sealing sheet for covering the rim of the container body and the space, wherein the sealing sheet corresponding to the space has a plurality of apertures.

20. The food/drink container of claim 19, further comprising a sealing layer for covering the plurality of apertures of the sealing sheet.
